(19) United States
(12) Patent Application Publication
(10) Pub. No.: US 2024/0369917 A1
(43) Pub. Date: Nov. 7, 2024

(54) MEDICAL IMAGE PROCESSING DEVICE, TREATMENT SYSTEM, MEDICAL IMAGE PROCESSING METHOD, AND STORAGE MEDIUM

(71) Applicants: TOSHIBA ENERGY SYSTEMS & SOLUTIONS CORPORATION, Kawasaki-shi (JP); National Institutes for Quantum Science and Technology, Chiba-shi (JP)

(72) Inventors: Keiko OKAYA, Setagaya Tokyo (JP); Yasuhiro SOEKAWA, Hachioji Tokyo (JP); Yasushi ISEKI, Yokohama Kanagawa (JP); Shinichiro MORI, Chiba-shi (JP)

(73) Assignees: TOSHIBA ENERGY SYSTEMS & SOLUTIONS CORPORATION, Kawasaki-shi (JP); National Institutes for Quantum Science and Technology, Chiba-shi (JP)

(21) Appl. No.: 18/772,508

(22) Filed: Jul. 15, 2024

Related U.S. Application Data
(63) Continuation of application No. PCT/JP2023/005127, filed on Feb. 15, 2023.

(30) Foreign Application Priority Data
Mar. 16, 2022 (JP) 2022-041518

(51) Int. Cl. G03B 42/02; A61B 6/00; A61N 5/10

(52) U.S. Cl. CPC G03B 42/026; A61B 6/485; A61N 5/10

(57) ABSTRACT

A medical image processing device according to an embodiment has a first image acquirer, a second image acquirer, a shifter, and a display controller. The first image acquirer acquires a first three-dimensional fluoroscopic image that is a three-dimensional fluoroscopic image of a patient captured in a patient treatment planning stage. The second image acquirer acquires a second three-dimensional fluoroscopic image that is a three-dimensional fluoroscopic image of the patient captured in a patient treatment stage. The shifter adds a predetermined amount to a photographing center position of the second three-dimensional fluoroscopic image to shift a display center position of the second three-dimensional fluoroscopic image. The display controller causes a display device to display a cross-sectional image of the second three-dimensional fluoroscopic image so that the display center position of the second three-dimensional fluoroscopic image is a center of a display region in a positioning stage of the patient treatment stage.

### CROSS-REFERENCE TO RELATED APPLICATION

The present application claims priority based on Japanese Patent Application No. 2022-041518 filed Mar. 16, 2022 and PCT/JP2023/005127 filed Feb. 15, 2023, the contents of which are incorporated herein by reference.

### FIELD

Embodiments of the present invention relate to a medical image processing device, a treatment system, a medical image processing method, and a storage medium.

### BACKGROUND

Radiation therapy is a treatment method for destroying a lesion in a body of a patient by irradiating the lesion with radiation. In this case, it is necessary for radiation to be accurately radiated to a position of the lesion. This is because a normal tissue inside a body of the patient may be influenced when the normal tissue is irradiated with the radiation. To this end, when the radiation therapy is performed, first, computed tomography (CT) is performed in advance and a position of the lesion in the body of the patient is ascertained in three dimensions in a treatment planning stage. A direction of radiation irradiation or intensity of the irradiation radiation is planned to reduce radiation toward normal tissue, based on the ascertained position of the lesion. Thereafter, in a treatment stage, a position on the patient is matched with that of the patient in the treatment planning stage, and the lesion is irradiated with radiation according to an irradiation direction or irradiation intensity planned in the treatment planning stage.

In aligning the patient in the treatment stage, three-dimensional CT data is virtually disposed in a treatment room, and a position of a movable bed in the treatment room is adjusted so that a position of the patient actually laid on a bed matches a position of the three-dimensional CT data. More specifically, two images including a three-dimensional CT image of the patient photographed in a state where the patient lies on the bed and a three-dimensional CT image captured at the time of treatment planning are collated (3D-3D positioning) so that a deviation in a position of the patient between the two images is obtained. The bed is moved based on the deviation in the position of the patient obtained through the image collation, a position of a lesion or bone inside a body of the patient is aligned with that at the time of treatment planning so that the positioning is approved, and the lesion is irradiated with radiation. When a CT photographing position is different from a radiation irradiation position, two images including an X-ray fluoroscopic image of the inside of the body of the patient and a digitally reconstructed radiograph (DRR) image obtained by virtually reconstructing the X-ray fluoroscopic image from the three-dimensional CT image captured at the time of treatment planning are further collated with each other (3D-2D positioning) when necessary so that positioning is approved in order to move the bed from the CT photographing position to the irradiation position, and the lesion is irradiated with the radiation.

However, center positions of images to be displayed may be different between the three-dimensional CT image captured in the treatment planning stage and the three-dimensional CT images captured in the treatment stage, and convenience may be low for the radiation therapist who performs positioning.

### BRIEF DESCRIPTION OF THE DRAWINGS

FIG. 1 is a block diagram showing a schematic configuration of a treatment system including a medical image processing device 100 of the embodiment.

FIG. 2 is a block diagram mainly showing a schematic configuration of the medical image processing device 100 of the embodiment.

FIG. 3 is a diagram showing a difference between a first image captured in a treatment planning stage and a second image captured in a treatment stage.

FIG. 4 is a diagram showing an example of a method in which an image position shifter 130 shifts a display center position of the second image.

FIG. 5 is a diagram showing an example of a method in which the image position shifter 130 shifts a display center position of the first image.

FIG. 6 is a diagram showing an example of a UI that receives a designation of a display center position.

FIG. 7 is a diagram showing an example of a second image captured by a CBCT device.

FIG. 8 is a flowchart showing an example of a flow of processing that is executed by the medical image processing device 100.

### DETAILED DESCRIPTION

Hereinafter, a medical image processing device, a treatment system, a medical image processing method, and a storage medium of the embodiment will be described with reference to the drawings.

A medical image processing device according to an embodiment has a first image acquirer, a second image acquirer, a shifter, and a display controller. The first image acquirer acquires a first three-dimensional fluoroscopic image that is a three-dimensional fluoroscopic image of a patient captured in a patient treatment planning stage. The second image acquirer acquires a second three-dimensional fluoroscopic image that is a three-dimensional fluoroscopic image of the patient captured in a patient treatment stage. The shifter adds a predetermined amount to a photographing center position of the second three-dimensional fluoroscopic image to shift a display center position of the second three-dimensional fluoroscopic image. The display controller causes a display device to display a cross-sectional image of the second three-dimensional fluoroscopic image so that the display center position of the second three-dimensional fluoroscopic image is a center of a display region in a positioning stage of the patient treatment stage.

### [Overall Configuration]

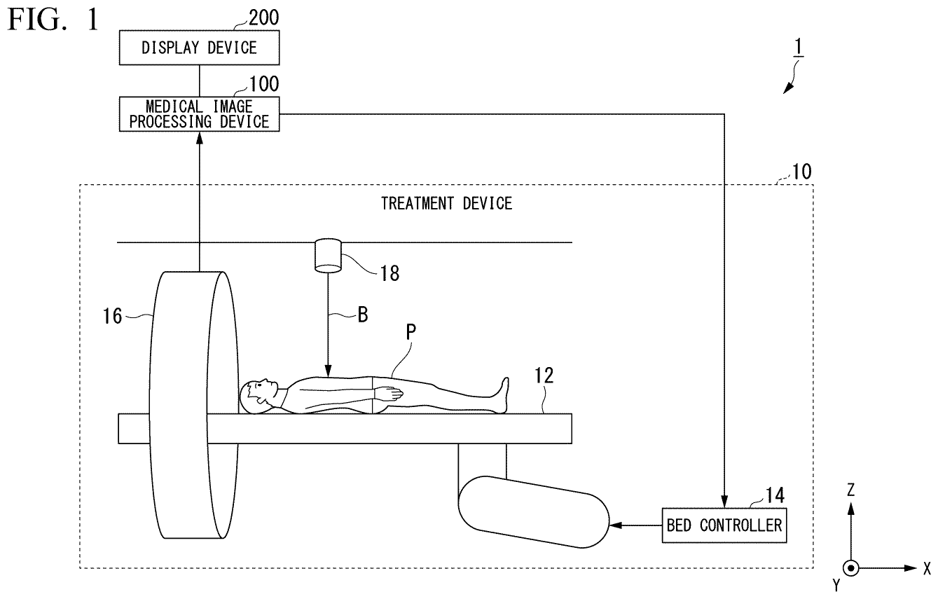

FIG. 1 is a block diagram showing a schematic configuration of a treatment system including a medical image processing device 100 of an embodiment. The treatment system 1 includes, for example, a treatment device 10, a medical image processing device 100, and a display device 200. The treatment device 10 includes, for example, a bed 12, a bed controller 14, a computed tomography (CT) device 16 (hereinafter referred to as a “CT photographing device 16”), and a treatment beam irradiation gate 18.

The bed 12 is a movable treatment table that fixes a subject (patient) P who receives treatment using radiation, for example, in a lying state by a fixing tool or the like. The bed 12 moves in a state where the patient P is fixed inside the annular CT photographing device 16 having an opening according to the control of the bed controller 14. The bed controller 14 controls a translation mechanism and a rotation mechanism provided in the bed 12 to perform positioning of the patient P fixed to the bed 12 according to a movement amount signal output from the medical image processing device 100. The translation mechanism can drive the bed 12 in three axial directions, and the rotation mechanism can rotate the bed 12 around three axes. That is, the bed controller 14 controls, for example, the translation mechanism and the rotation mechanism of the bed 12 to move the bed 12 with six degrees of freedom. A degree of freedom at which the bed controller 14 controls the bed 12 may not be the six degrees of freedom, and may be a degree of freedom (for example, four degrees of freedom) lower than the six degrees of freedom or a degree of freedom (for example, eight degrees of freedom) higher than the six degrees of freedom. The bed 12 is installed so that a position where photographing using the CT photographing device 16 is executed and a position where radiation of a treatment beam B using the treatment beam irradiation gate 18 is performed can be moved when the positions are different.

The CT photographing device 16 is an imaging device for performing three-dimensional computed tomography. The CT photographing device 16 includes a plurality of radiation sources disposed inside a circular (gantry) opening, and radiates radiation for seeing through the inside of the body of the patient P from each radiation source. That is, the CT photographing device 16 radiates the radiation from a plurality of positions around the patient P. The radiation radiated from each radiation source in the CT photographing device 16 is, for example, X-rays. The CT photographing device 16 detects the radiation radiated from the corresponding radiation source, passing through the inside of the body of the patient P, and reaching a plurality of radiation detectors disposed inside the circular opening using the radiation detectors. The CT photographing device 16 generates a CT image of the inside of the body of the patient P based on the magnitude of the energy of the radiation detected by each radiation detector. The CT image of the patient P generated by the CT photographing device 16 is a three-dimensional digital image in which a magnitude of a degree of radiation attenuation at each location inside the body is expressed as a digital value. The CT photographing device 16 outputs the generated CT image to the medical image processing device 100. The photography of the inside of the body of the patient P in the CT photographing device 16, that is, the irradiation of the radiation from each radiation source or the generation of the CT image based on the radiation detected by each radiation detector is controlled, for example, by a photography controller (not shown).

The treatment beam irradiation gate 18 radiates radiation for destroying a tumor (lesion), which is a treatment target site present in the body of the patient P, as the treatment beam B. The treatment beam B is, for example, an X-ray, a y ray, an electron beam, a proton beam, a neutron beam, or a heavy particle beam. The treatment beam B is radiated linearly from the treatment beam irradiation gate 18 to the patient P (more specifically, the tumor in the body of the patient P). The irradiation of the treatment beam B at the treatment beam irradiation gate 18 is controlled by, for example, a treatment beam irradiation controller (not shown). In the treatment system 1, the treatment beam irradiation gate 18 is an example of an “irradiator.”

In the radiation therapy, the treatment plan is made in a situation in which a treatment room is simulated. That is, in the radiation therapy, an irradiation direction, intensity, or the like when the patient P is irradiated with the treatment beam B is planned through a simulation of a state where the patient P is laid on the bed 12 in the treatment room. Specifically, an irradiation target location is specified by a doctor for the CT image, or such processing is performed automatically.

FIG. 1 shows a configuration of the treatment device 10 including the CT photographing device 16 and one fixed treatment beam irradiation gate 18, but the configuration of the treatment device 10 is not limited to the above-described configuration. For example, the treatment device 10 may be configured to include a CT photographing device having a configuration in which a set of a radiation source and a radiation detector rotate inside the circular opening, and a photographing device that generates a three-dimensional image of the inside of the body of the patient P, such as a cone-beam (CB) CT device, a magnetic resonance imaging (MRI) device, or an ultrasonic diagnostic device, instead of the CT photographing device 16. For example, the treatment device 10 may be configured to include a plurality of treatment beam irradiation gates and, for example, may further include a treatment beam irradiation gate that irradiates the patient P with a treatment beam in a horizontal direction. For example, the treatment device 10 may be configured to irradiate the patient P with a treatment beam from various directions by rotating around the patient P, such as by rotating one treatment beam irradiation gate 18 shown in FIG. 1 360 degrees around a rotation axis in a horizontal direction X shown in FIG. 1. For example, the treatment device 10 may be configured to include one or a plurality of imaging devices configured of a pair of a radiation source and a radiation detector instead of the CT photographing device 16, and the imaging device may be configured to rotate 360 degrees around the rotation axis in the horizontal direction X shown in FIG. 1 to photograph the inside of the body of the patient P from various directions. This configuration is called a rotating gantry type treatment device. In this case, for example, one treatment beam irradiation gate 18 shown in FIG. 1 may be configured to rotate simultaneously around the same rotation axis as the imaging device. Furthermore, in FIG. 1, the CT photographing device 16 and the treatment beam irradiation gate 18 are installed at close positions, but the CT photographing device 16 and the treatment beam irradiation gate 18 may be installed at positions apart from each other, and the positions of the CT photographing device 16 and the treatment beam irradiation gate 18 may be movable relative to each other by the bed 12 on which the patient P is laid.

The medical image processing device 100 performs processing for aligning the position of the patient P when performing radiation therapy based on the CT image output by the CT photographing device 16. More specifically, the medical image processing device 100 performs processing (3D-3D positioning) for aligning the position of the tumor or tissue present inside the body of the patient P, based on a CT image of the patient P captured before the radiation therapy is performed, for example, in a treatment planning stage, and a current CT image of the patient P captured by the CT photographing device 16 in a treatment stage in which the radiation therapy is performed (treatment stage). The medical image processing device 100 outputs the movement amount signal for moving the bed 12 to the bed controller 14 to align the position of the patient P with the same body position as in the time of the treatment plan. That is, the medical image processing device 100 outputs to the bed controller 14 a movement amount signal for moving the patient P to a position and at a posture where the tumor or tissue to be treated can be appropriately irradiated with the treatment beam B in the radiation therapy.

The display device 200 displays images for presenting various types of information in the treatment system 1 to a radiation therapist (such as a doctor) who uses the treatment system 1, including during positioning of the patient P in the medical image processing device 100. The display device 200 displays various images such as the CT image or X-ray fluoroscopic image output by the medical image processing device 100, or an image obtained by superimposing various types of information on these images. The display device 200 is, for example, a display device such as a liquid crystal display (LCD). The radiation therapist (such as a doctor) can obtain information when the radiation therapy is performed using the treatment system 1 by visually confirming the image displayed on the display device 200. The treatment system 1 may be configured to include a user interface such as an operator (not shown) operated by the radiation therapist (such as a doctor), and to allow various functions executed by the treatment system 1 to be manually operated.

The medical image processing device 100, the display device 200, and the bed controller 14 or the CT photographing device 16 included in the treatment device 10 may be connected by wires, or may be connected wirelessly, for example, via a local area network (LAN) or a wide area network (WAN).

### [Medical Image Processing Device]

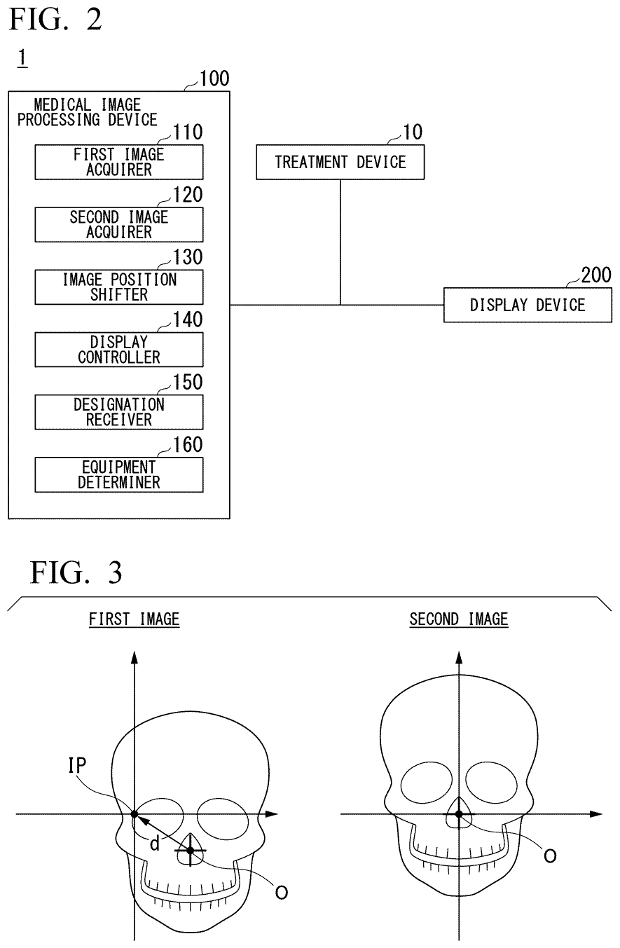

Hereinafter, the medical image processing device 100 of the embodiment will be described. FIG. 2 is a block diagram mainly showing a schematic configuration of the medical image processing device 100 of the embodiment. The medical image processing device 100 includes, for example, a first image acquirer 110, a second image acquirer 120, an image position shifter 130, a display controller 140, a designation receiver 150, and an equipment determiner 160.

Some or all of components of the medical image processing device 100 are realized by, for example, a hardware processor such as a central processing unit (CPU) executing a program (software). Some or all of these components may be realized by hardware (circuit; including circuitry) such as a large scale integration (LSI), an application specific integrated circuit (ASIC), a field-programmable gate array (FPGA), or a graphics processing unit (GPU), or may be realized by collaboration between software and hardware. Some or all of functions of these components may be realized by a dedicated LSI. The program may be stored in a storage device (a storage device including a non-transitory storage medium) such as a read only memory (ROM), a random access memory (RAM), a hard disk drive (HDD), or a flash memory included in the medical image processing device 100 in advance, or may be stored in a removable storage medium (non-transitory storage medium) such as a DVD or CD-ROM and installed in the HDD or flash memory included in the medical image processing device 100 when the storage medium is mounted on a drive device included in the medical image processing device 100. The program may be downloaded from another computer device via a network and installed in the HDD or flash memory included in the medical image processing device 100.

The first image acquirer 110 acquires a first image regarding the patient P before treatment and a parameter associated with the first image. The first image is, for example, a three-dimensional CT image representing a three-dimensional shape of the inside of the body of the patient P, which is photographed by the CT photographing device 16 in the treatment planning stage when the radiation therapy is performed. The first image is used for a determination of a direction (a path including an inclination, a distance, or the like) or intensity of the treatment beam B to be radiated to the patient P in the radiation therapy. The first image is displayed by the display device 200 with a region of interest ROI when the radiation therapy is performed on the patient P as a display center position. The region of interest ROI can also be expressed as a position IP (isocenter position) of a region that is irradiated most intensively with radiation at the time of the radiation therapy. The first image is an example of a “first three-dimensional fluoroscopic image.”

Generally, the captured CT image is displayed by the display device 200 with a photographing center position O (CT isocenter position) as the display center position. However, in the CT image captured in the treatment planning stage, then, the region of interest ROI is designated by the radiation therapist, and an isocenter position IP is set as the display center position. In this case, a (three-dimensional) displacement amount d of the isocenter position IP designated in the treatment planning stage, with the photographing center position O at the time of capturing the first image as a reference, is stored as the parameter associated with the first image.

The second image acquirer 120 acquires a second image of the patient P immediately before the start of the radiation therapy. The second image is, for example, a three-dimensional CT image representing the three-dimensional shape of the inside of the body of the patient P photographed by the CT photographing device 16 in order to align the body position of the patient P at the time of irradiation with the treatment beam B in the radiation therapy (that is, to perform positioning). That is, the second image is an image captured by the CT photographing device 16 immediately before the treatment beam B is radiated from the treatment beam irradiation gate 18. In this case, the first image and the second image are captured with different times, devices, and places, but the second image is captured at substantially the same body position as that when the first image has been captured. For example, when a mark is written on the fixing tool that fixes the patient P to the bed in alignment with a laser of the CT device at the time of capturing the first image, the mark on the fixing tool is aligned with the laser of the CT device at the time of capturing the second image, and then, photographing is performed, the photographing centers O of the first image and the second image become substantially the same position of the patient. For example, when a position of the bed relative to the CT device when the first image is captured is recorded, and the bed is moved to the recorded position when the second image is captured, the photographing centers O of the first image and the second image become substantially the same position of the patient. The second image is displayed by the display device 200 with the photographing center position O (CT isocenter position) at the time of photographing as the display center position. The second image is an example of a “second three-dimensional fluoroscopic image”.

FIG. 3 is a diagram showing a difference between the first image captured in the treatment planning stage and the second image captured in the treatment stage. In FIG. 3, a left image shows the first image captured in the treatment planning stage, and a right image shows the second image captured in the treatment stage. As shown in FIG. 3, the first image is displayed by the display device 200 with the isocenter position IP as the display center position, while the second image is displayed by the display device 200 with the photographing center position O as the display center position. In other words, since the display center position of the first image is different from the display center position of the second image, it may be less convenient for the radiation therapist to collate the first image with the second image.

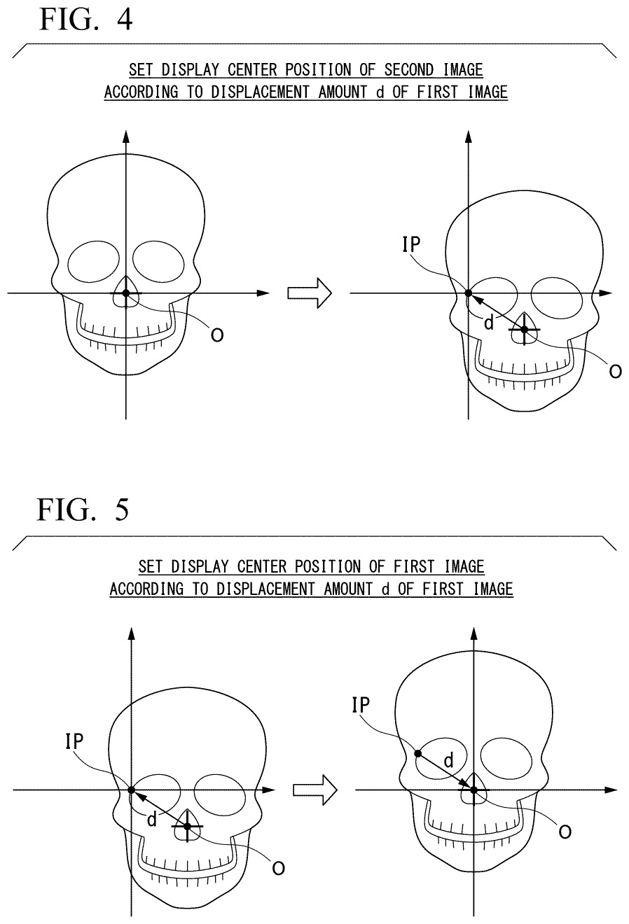

In light of the above circumstances, the image position shifter 130 adds a predetermined amount to the photographing center position O of the second image to shift the display center position of the second image. FIG. 4 is a diagram showing an example of a method in which the image position shifter 130 shifts the display center position of the second image. In FIG. 4, a left image shows the second image before the display center position is shifted, and an image on the right represents the second image after the display center position is shifted. As shown in FIG. 4, the image position shifter 130, for example, adds the displacement amount d of the isocenter position IP designated in the treatment planning stage, with the photographing center position O of the first image as a reference, to the photographing center position O of the second image to shift the display center position of the second image. The display controller 140 causes the display device 200 to display a cross-sectional image of the second image so that the shifted display center position of the second image is the center of the display region. This makes it possible to substantially match the display center positions of the first image and the second image with each other and to improve the convenience for the radiation therapist who performs the positioning.

In this case, the medical image processing device 100 may simultaneously output a movement amount signal for moving the bed 12 by a distance equivalent to the displacement amount d to the bed controller 14, and the bed controller 14 may control the translation mechanism and the rotation mechanism of the bed 12 to move the bed 12 according to the received movement amount signal. This makes it possible to further improve the convenience for the radiation therapist who performs the positioning. Alternatively, when the shifted second image is displayed, the displacement amount d may be displayed as a treatment table movement amount, a positioning calculation using the first image and the second image is performed, rewriting to the calculated treatment table movement amount is then performed, and a movement amount signal may be output to the bed controller 14.

In another embodiment, the image position shifter 130 may execute 3D-3D positioning between the first image captured in the treatment planning stage and the second image captured in the treatment stage using positioning software (not shown) installed in the medical image processing device 100, to add a displacement amount between the first image and the second image specified by the 3D-3D positioning to the photographing center position O of the second image and shift the display center position of the second image.

In yet another embodiment, the image position shifter 130 may shift the display center position of the first image without shifting the display center position of the second image to match the display center positions of the first image and the second image. FIG. 5 is a diagram showing an example of a method in which the image position shifter 130 shifts the display center position of the first image. In FIG. 5, a left image shows the first image before the display center position is shifted, and a right image shows the first image after the display center position is shifted. As shown in FIG. 5, the image position shifter 130, for example, subtracts the displacement amount d of the isocenter position IP designated in the treatment planning stage, with the photographing center position O of the first image as a reference, from the photographing center position O of the first image (adds displacement amount-d) to shift the display center position of the first image. The display controller 140 causes the display device 200 to display the cross-sectional image of the first image so that the display center position of the shifted first image is the center of the display region. Thus, it is possible to substantially match the display center positions of the first image and the second image and improve convenience for the radiation therapist who performs the positioning.

The designation receiver 150 provides the display device 200 with a user interface (UI) for designating whether the display center position of the second image is set as the isocenter position IP or the photographing center position O, and receives a designation from the radiation therapist. The display controller 140 sets the isocenter position IP or the photographing center position O as the display center position according to the designation from the radiation therapist to cause the display device 200 to display the cross-sectional image of the second image.

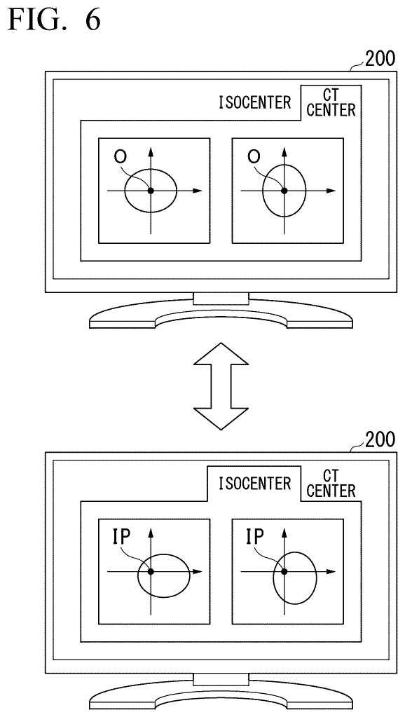

FIG. 6 is a diagram showing an example of a UI that receives the designation of the display center position. FIG. 6 shows an example in which the designation of the display center position is received by tab switching. As shown in FIG. 6, the designation receiver 150 provides, for example, an isocenter tab and a CT center tab. When the radiation therapist selects the isocenter tab, the designation receiver 150 determines that the display center position of the second image is to be set as the isocenter position IP, and the display controller 140 causes the display center position of the second image to be shifted by the displacement amount d and causes the display device 200 to display the cross-sectional image of the second image. On the other hand, when the radiation therapist selects the CT center tab, the designation receiver 150 causes the display device 200 to display the cross-sectional image of the second image without shifting the display center position of the second image. This makes it possible to further improve the convenience for the radiation therapist who performs the positioning. In FIG. 6, as an example, the display device 200 displays two cross-sectional images when the second image which is a three-dimensional image is viewed in two directions, but may be caused to display only a cross-sectional image from one direction or cross-sectional images from three or more directions. Display switching may be performed by buttons, check boxes, or a software setting instead of tab switching.

In the description, it is assumed that an initial display center position at the time of capturing the second image is the photographing center position O (CT isocenter position). This is because, even when CT photographing is performed with a region to be irradiated in the radiation therapy as a center, it is difficult to capture the CT image with the isocenter position IP as the display center position in a case where a gantry diameter of the CT photographing device 16 is small. On the other hand, when the CT photographing device 16 is a CBCT device, or when the gantry diameter of the CT photographing device 16 is large, it is possible to capture the CT image with the isocenter position IP as the display center position.

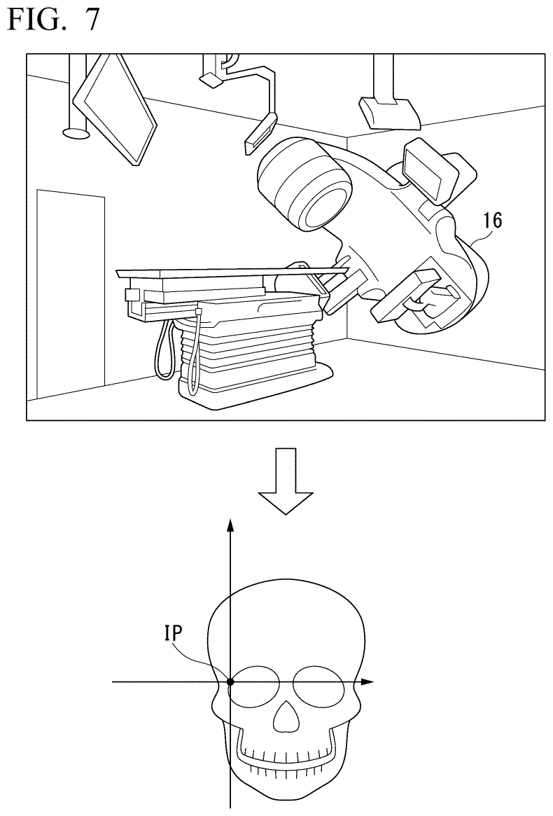

FIG. 7 is a diagram showing an example of the second image that is captured by the CBCT device. In FIG. 7, a left image shows a state where the second image is captured by the CT photographing device 16, which is the CBCT device, and a right image shows the second image that is captured by the CT photographing device 16. As shown in FIG. 7, the gantry diameter of the CT photographing device 16, which is a CBCT device, is larger than that of the CT photographing device 16 shown in FIG. 1, and the CT photographing device 16 can set the isocenter position IP as the display center position to capture the CT image.

With the above circumstances as a background, the equipment determiner 160 determines whether the second image has been captured by the CT photographing device 16, which is a CBCT device, or whether the gantry diameter of the CT photographing device 16 is equal to or greater than the predetermined value. When a determination is made that the second image has been captured by the CT photographing device 16, which is a CBCT device, or that the gantry diameter is equal to or greater than the predetermined value, the display controller 140 sets the isocenter position IP as the display center position and causes the display device 200 to display the cross-sectional image of the second image, without shifting the display center position of the second image. On the other hand, when a determination is made that the second image is not captured by the CT photographing device 16, which is a CBCT device, and the gantry diameter is smaller than the predetermined value, the display controller 140 causes the display center position of the second image to be shifted by the displacement amount d, and causes the display device 200 to display the cross-sectional image of the second image. Such display switching using the equipment determiner 160 and the display controller 140 is realized, for example, by storing a setting file specifying that a display position is switched depending on a model and gantry diameter of the CT photographing device 16 in the medical image processing device 100, and referring to the setting file when the second image is acquired. This means the display switching according to the above-described software setting. This makes it possible to further improve the convenience for the radiation therapist who performs the positioning.

The second image acquirer 120 may, for example, acquire identification information of the CT photographing device 16 that has captured the second image together with the second image, and the equipment determiner 160 may determine whether the second image has been captured by the CT photographing device 16 that is the CBCT device, based on the acquired identification information. Further, for example, the equipment determiner 160 may refer to tag information assigned to the second image to make a determination, based on whether the tag information indicates that the second image has been captured by the CT photographing device 16 that is a CBCT device.

As yet another aspect, when a photographing position where the CT photographing device 16 captures the second image is different from an irradiation position where the patient is irradiated with the treatment beam B, the display controller 140 may acquire position information of the bed 12 at a point in time when the second image has been captured, and determine whether or not to shift the position of the second image depending on whether the position information is the irradiation position. More specifically, when the position information of the bed 12 indicates the irradiation position, the display controller 140 may determine that the CT photographing device 16 is the CBCT device, and cause the display device 200 to display the cross-sectional image of the second image without shifting the display center position of the second image. On the other hand, when the position information of the bed 12 does not indicate the irradiation position, the display controller 140 may determine that the CT photographing device 16 is not the CBCT device, shift the display center position of the second image by the displacement amount d, and cause the cross-sectional image of the second image to be displayed on the display device 200.

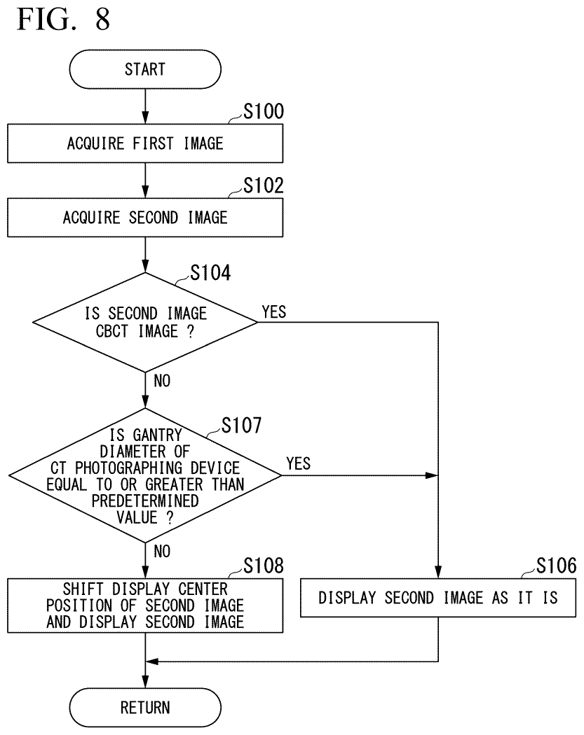

Next, a flow of processing that is executed by the medical image processing device 100 will be described with reference to FIG. 8. FIG. 8 is a flowchart showing an example of the flow of processing that is executed by the medical image processing device 100.

First, the first image acquirer 110 acquires a first image captured by the CT photographing device 16 in the treatment planning stage (step S100). Next, the second image acquirer 120 acquires a second image captured by the CT photographing device 16 in a patient positioning stage of the treatment stage (step S102). Next, the equipment determiner 160 determines whether the second image acquired by the second image acquirer 120 has been captured by the CT photographing device 16, which is a CBCT device (step S104).

When a determination is made that the second image acquired by the second image acquirer 120 has been captured by the CT photographing device 16, which is a CBCT device (Yes in step S104), the display controller 140 sets the isocenter position IP as the display center position and causes the display device 200 to display the cross-sectional image of the second image, without shifting the display center position of the second image (step S106).

On the other hand, when a determination is not made that the second image acquired by the second image acquirer 120 has been captured by the CT photographing device 16, which is a CBCT device (No in step S104), the display controller 140 determines whether the gantry diameter of the CT photographing device 16 is equal to or greater than the predetermined value (step S107). When a determination is made that the gantry diameter of the CT photographing device 16 is equal to or greater than the predetermined value (Yes in step S107), the display controller 140 executes the processing of step S106. On the other hand, when a determination is made that the gantry diameter of the CT photographing device 16 is smaller than the predetermined value (No in step S107), the display controller 140 causes the display center position of the second image to be shifted by the displacement amount d, and causes the display device 200 to display the cross-sectional image of the second image (step S108). Accordingly, the processing of this flowchart ends.

According to at least one embodiment described above, a displacement amount of the isocenter position with the photographing center position of the CT image captured in the treatment planning stage as a reference is added to the photographing center position of the CT image captured in the treatment stage, so that the display center positions of these CT images are substantially matched with each other and the CT image captured in the treatment stage is displayed. This makes it possible to improve the convenience for the radiation therapist who performs the positioning.

In the embodiment, as an example, a case where one medical image processing device 100 can be used simultaneously for processing for a plurality of types of CT photographing devices 16 has been described, but the present invention is not limited to such a configuration. For example, in a facility (such as a hospital) where the medical image processing device 100 is installed, when only one type of CT photographing device 16 (such as a CT device or a CBCT device) is used, the medical image processing device 100 may be set so that the medical image processing device 100 can be used exclusively for processing for the single type of CT photographing device 16, by software at the time of shipment. With such a configuration, it is also possible to improve the convenience for the radiation therapist who performs the positioning according to a type of CT photographing device 16 to which the medical image processing device 100 is applied.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing device comprising:
a first image acquirer configured to acquire a first three-dimensional fluoroscopic image, the first three-dimensional fluoroscopic image being a three-dimensional fluoroscopic image of a patient captured in a patient treatment planning stage;
a second image acquirer configured to acquire a second three-dimensional fluoroscopic image, the second three-dimensional fluoroscopic image being a three-dimensional fluoroscopic image of the patient captured in a patient treatment stage;
a shifter configured to add a predetermined amount to a photographing center position of the second three-dimensional fluoroscopic image to shift a display center position of the second three-dimensional fluoroscopic image; and
a display controller configured to cause a display device to display a cross-sectional image of the second three-dimensional fluoroscopic image so that the display center position of the second three-dimensional fluoroscopic image is a center of a display region in a positioning stage of the patient treatment stage.

2. The medical image processing device according to claim 1, wherein the predetermined amount is a displacement amount of an isocenter position designated in the treatment planning stage, with the photographing center position of the first three-dimensional fluoroscopic image as a reference.

3. The medical image processing device according to claim 1, wherein the predetermined amount is a displacement amount obtained by performing 3D-3D positioning between the first three-dimensional fluoroscopic image and the second three-dimensional fluoroscopic image.

4. The medical image processing device according to claim 1, further comprising:
a receiver configured to receive a designation as to whether or not to cause the display device to display the cross-sectional image of the second three-dimensional fluoroscopic image so that the display center position shifted by the shifter is the center of a display region.

5. The medical image processing device according to claim 1, further comprising:
a determiner configured to determine whether or not the second three-dimensional fluoroscopic image has been captured by a predetermined type of computed tomography device, wherein
the shifter is configured not to shift the display center position of the second three-dimensional fluoroscopic image when the determiner determines that the second three-dimensional fluoroscopic image has been captured by a predetermined type of computed tomography device.

6. The medical image processing device according to claim 5, wherein the determiner is configured to determine whether the second three-dimensional fluoroscopic image has been captured by a predetermined type of computed tomography device, based on identification information of equipment serving as an acquisition source from which the second three-dimensional fluoroscopic image has been acquired.

7. The medical image processing device according to claim 5, wherein the determiner is configured to determine whether the second three-dimensional fluoroscopic image has been captured by a predetermined type of computed tomography device, based on tag information assigned to the second three-dimensional fluoroscopic image.

8. The medical image processing device according to claim 1, wherein the display controller is configured to cause the display device to display a movement amount of a bed on which the patient is laid, which corresponds to the shifted amount, when causing the display device to display the cross-sectional image of the second three-dimensional fluoroscopic image of which the display center position has been shifted.

9. The medical image processing device according to claim 5, wherein
the determiner is configured to determine whether or not a position of a bed on which the patient is laid at a point in time when the second three-dimensional fluoroscopic image is captured is at an irradiation position at which the patient is irradiated with radiation when a photographing position at which the second three-dimensional fluoroscopic image is captured is different from the irradiation position, and
the display controller is configured not to shift the display center position of the second three-dimensional fluoroscopic image when a determination is made that the position of the bed is at the irradiation position, and to shift the display center position of the second three-dimensional fluoroscopic image when a determination is made that the position of the bed is not at the irradiation position.

10. A medical image processing device comprising:
a first image acquirer configured to acquire a first three-dimensional fluoroscopic image, the first three-dimensional fluoroscopic image being a three-dimensional fluoroscopic image of a patient captured in a patient treatment planning stage;
a second image acquirer configured to acquire a second three-dimensional fluoroscopic image, the second three-dimensional fluoroscopic image being a three-dimensional fluoroscopic image of the patient captured in a patient treatment stage;
a shifter configured to add a predetermined amount to a photographing center position of the first three-dimensional fluoroscopic image to shift a display center position of the first three-dimensional fluoroscopic image; and
a display controller configured to cause a display device to display a cross-sectional image of the first three-dimensional fluoroscopic image so that the display center position of the first three-dimensional fluoroscopic image is a center of a display region in a positioning stage of the patient treatment stage.

11. A treatment system comprising:
the medical image processing device according to claim 1; and
a treatment device including an irradiator configured to irradiate the patient with radiation, an imaging device configured to capture the first three-dimensional fluoroscopic image and the second three-dimensional fluoroscopic image, a bed on which the patient is laid and fixed, and a bed controller configured to perform control to move the bed by the predetermined amount.

12. A medical image processing method comprising:
acquiring, by a computer, a first three-dimensional fluoroscopic image, the first three-dimensional fluoroscopic image being a three-dimensional fluoroscopic image of a patient captured in a patient treatment planning stage;
acquiring, by the computer, a second three-dimensional fluoroscopic image, the second three-dimensional fluoroscopic image being a three-dimensional fluoroscopic image of the patient captured in a patient treatment stage;
adding, by the computer, a predetermined amount to a photographing center position of the second three-dimensional fluoroscopic image to shift a display center position of the second three-dimensional fluoroscopic image; and
causing, by the computer, a display device to display a cross-sectional image of the second three-dimensional fluoroscopic image so that the display center position of the second three-dimensional fluoroscopic image is a center of a display region in a positioning stage of the patient treatment stage.

13. A computer-readable non-transitory storage medium storing a program for causing a computer to:
acquire a first three-dimensional fluoroscopic image, the first three-dimensional fluoroscopic image being a three-dimensional fluoroscopic image of a patient captured in a patient treatment planning stage;
acquire a second three-dimensional fluoroscopic image, the second three-dimensional fluoroscopic image being a three-dimensional fluoroscopic image of the patient captured in a patient treatment stage;
add a predetermined amount to a photographing center position of the second three-dimensional fluoroscopic image to shift a display center position of the second three-dimensional fluoroscopic image; and
cause a display device to display a cross-sectional image of the second three-dimensional fluoroscopic image so that the display center position of the second three-dimensional fluoroscopic image is a center of a display region in a positioning stage of the patient treatment stage.
